(19) United States
(12) Patent Application Publication
(10) Pub. No.: US 2004/0219612 A1
(43) Pub. Date: Nov. 4, 2004

(54) Methods for detecting intracellular defensins in various leukocyte subpopulations

(75) Inventor: Sybil S. D'Costa, Pembroke Pines, FL (US)

Correspondence Address: BECKMAN COULTER, INC., P.O. BOX 169015, MAIL CODE 32-A02, MIAMI, FL, 33116-9015, US

(73) Assignee: Beckman Coulter, Inc., Fullerton, CA

(21) Appl. No.: 10/428,870

(22) Filed: May 2, 2003

(51) Int. Cl. G01N 33/567

(52) U.S. Cl. 435/7.21

(57) ABSTRACT

The present invention relates to methods and kits for determining intracellular defensin expression in specific leukocyte subpopulations by flow cytometry. Specific leukocyte subpopulations may be identified by measuring the signals generated from one or more cell-distinguishing antibodies bound to cells in a sample. Intracellular defensin expression in specific leukocyte subpopulations may be determined by measuring the signals generated from one or more defensin-specific antibodies bound to defensin inside the cells. These methods may be used to generate an intracellular defensin expression profile which may be useful for diagnosing and treating diseases and illnesses.

### FIELD OF THE INVENTION

The present invention relates to a method and a kit for determining leukocyte subpopulations expressing intracellular defensin. The present invention also relates to a method for determining a defensin-expression profile of leukocyte subpopulations. The methods of the present invention may be useful for diagnosis and treatment of various diseases, infections, and illnesses.

### BACKGROUND OF THE INVENTION

Mammalian defensins are small antimicrobial peptides (3.5-4.5 kDa) that are characterized by the presence of six cysteine residues. These cysteine residues form three disulfide bonds whose ordered array determines whether these proteins are classified as α (alpha), β (beta), and the θ (theta) defensins.

To date, there are six known α defensins: the human neutrophil proteins (HNP 1-4) (Ganz et al., 1985, J. Clin. Invest. 76:1427-1435; Wilde et al. 1989, J. Biol. Chem. 264:11200-11203) and the human intestinal defensins (HD-5, HD-6) (Mallow et al., 1996, J. Biol. Chem. 271:4038-4045; Porter et al., 1997, Inf. Immun. 65:2389-2395). The α defensins have been found in primarily in granulocytes (neutrophils) (Ganz et al., 1995, Pharm. Therap. 66:191-205), but has also been detected in lymphocytes (NK cells, B cells, CD3 T cells) and monocytes (Agerberth et al., 2000, Blood 96:3086-3093; Klutt et al., 2000, Eur. J. Haematol. 64:114-120). Recently, α defensin has been detected in CD8+ T cells in long-term non-progressors infected with the human immunodeficiency virus 1 (HIV-1) (Zhang et al., 2002, Science 298:995-1000). However, expression of α defensin may wane over a period of time (Obata-Onai et al., 2002, Int. Immunol. 14:1085-1098).

β defensins were originally isolated from bovine neutrophils (Selsted et al., 1993, J. Biol. Chem. 268:6641-6648). Since then, four β defensins have been isolated from humans (HBD 1-4) (Bensch et al., 1995, FEBS Letters 368:331-335; Goldman et al., 1997, Cell 88:553-560; Cole et al., 1999, Infect. Immun. 67:3267-3275; Garcia et al., 2001, Cell Tissue Res. 306:257-264; Harder et al., 2001, J. Biol. Chem. 276:5707-5713; Garcia et al., 2001, FASEB J. 15:1819-1821). Human β defensins have so far been detected in monocytes, monocyte-derived macrophages, and monocyte-derived dendritic cells (Duits et al., 2002, Immunology 106:517-525). β defensins likely contribute to the host defense system of mucosal surfaces (Selsted et al., 1993, J. Biol. Chem. 268:6641-6648; Tang et al., 1993, J. Biol. Chem. 268:6649-6653; Harwig et al., 1994, FEBS Letters 342:281-285; Evans et al., 1994. J. Leuc. Biol. 56:661-665).

The theta defensins are mini circular trisulphide peptides, 18 amino acids in length. The theta defensins are expressed in monocytes and neutrophils of rhesus macaques (Tang et al., 1999, Science 286: 498-502). The cellular machinery that is required for the post translational processing of theta defensins is found in human leukocytes (Lenova et al., 2001, J Leukoc Biol 70: 461-464). Moreover, a pseudogene that encoded an antimicrobial peptide similar to the rhesus monkey theta defensin has been shown to be present in human bone marrow (Cole et al., 2002, PNAS 99:1813-1818). Although it is yet unknown whether the theta defensin protein is also made in human lymphocytes, in vitro assays have been shown that theta defensins are able to protect human CD4+ T cells from HIV infections (Cole et al., 2002, PNAS 99: 1813-1818) possibly by binding to the carbohydrate components of surface glycoproteins of HIV (Wang et al., 2003, J Immunol. 170 (9): 4708-4716).

The mechanism of antimicrobial activity of defensins is believed to be via selective membrane disruption. Antimicrobial activity is initiated by electrostatic interactions of defensin with negatively charged target cell surface molecules, followed by insertion of defensin into the cell membranes (Boman, 1995, Annu. Rev. Immunol. 13:61-92; Lehrer et al., 1993, Ann. Rev. of Immunol. 11:105-128). The antimicrobial spectrum of defensins includes gram positive and gram negative bacteria, mycobacteria, T. pallidum, many fungi, and some enveloped-viruses (Boman, 1995, Annu. Rev. Immunol. 13:61-92). Defensins also exert nonspecific cytotoxic activity against a wide range of normal and malignant cell targets. Defensin may additionally act as opsonins, inhibit signal transduction, block steroidogenesis, modulate cytokine production and adhesion molecule expression in human monocytes and endothelial cells, or act as selective chemoattractins for monocytes. Thus, defensins may play a considerable role in host cell defense, inflammation, and cytotoxicity in humans and are an integral part of the innate immune system and act as a bridge between innate and acquired immune response (Lehrer et al., 1993, Ann. Rev. of Immunol. 11:105-128, Lillard et al., 1999, Proc. Natl. Acad. Sci 96: 651-656).

Enhanced levels of secreted defensins have generally been associated with an active infection. Thus, measuring levels of extracellular defensins have provided an important tool in the diagnosis and subsequent treatment of these infections. The majority of the assays have measured secreted defensins via standard techniques of ELISA, radioimmunoassays, or determining mRNA transcript levels for defensin using RT-PCR. However, these methods do not allow for the identification of a specific cell type in the leukocyte population that is associated with an increased concentration of the defensin polypeptide.

Thus, there remains a need in the art for a method that determines defensin expression in specific leukocyte subpopulations.

### SUMMARY OF THE INVENTION

The present invention addresses these needs by providing a novel method for determining cell types expressing intracellular defensin. The method comprises combining a sample comprising a multiplicity of leukocyte subpopulations with at least one defensin-specific antibody and at least one cell-distinguishing antibody. At least one leukocyte subpopulation is identified by measuring the signal from the cell-distinguishing antibody bound to the cells. The signal from the defensin-specific antibody is measured in the leukocyte subpopulation identified. In an embodiment of the present invention, the defensin-specific antibody binds to defensin inside a cell of at least one leukocyte subpopulation and the cell-distinguishing antibody binds to the cell surface of at least one leukocyte subpopulation. The sample comprising a multiplicity of leukocyte subpopulations may be a whole blood sample or a purified sample such as one comprising lymphocytes, monocytes, neutrophils, and eosinophils.

The present invention also provides a method for determining a defensin-expression profile of a multiplicity of leukocyte subpopulations. The method comprises combining a sample comprising a multiplicity of leukocyte subpopulations with at least one defensin-specific antibody and identifying a multiplicity of leukocyte subpopulations. The signals from the defensin-specific antibody is measured in the leukocyte subpopulations to determine if the leukocyte populations express defensin and a defensin-expression profile is generated. In an embodiment of the present invention, the multiplicity of leukocyte subpopulations is identified according to light scatter without the use of a cell-distinguishing antibody. In another embodiment of the present invention, the multiplicity of leukocyte subpopulations is identified by combining the sample with at least one cell-distinguishing antibody. The method may further comprise determining other immunological parameters useful for identifying patterns or correlations of specific leukocyte subpopulations associated with intracellular defensin expression. For example, the method may further comprise detecting a cytokine-mediated response such as by determining an intracellular cytokine expression profile or a cytokine-binding profile. Other useful immunological profiles may include a peptide-MHC binding profile, an antigen-specific binding profile, a cell proliferation profile, or an immune activation profile of a multiplicity of leukocyte subpopulations.

The present invention further provides a kit for determining cell types expressing intracellular defensin or for determining a defensin-expression profile of a multiplicity of leukocyte subpopulations. The kit comprises at least one cell-distinguishing antibody and at least one defensin-specific antibody. In an embodiment of the present invention, the kit further comprises one or more of: a permeabilizing agent, an antibody to a cytokine, an antibody to a cytokine receptor, a cytokine that binds to a cytokine receptor, a peptide-MHC complex, an antigen recognized by a B cell, a reagent for detecting cell proliferation, or an antibody indicative of immune activation.

The present invention thus provides methods and kits for determining cell types expressing intracellular defensin and for determining the defensin-expression profile of a multiplicity of leukocyte subpopulations. Defensin expression may be associated with other immunological parameters.

### BRIEF DESCRIPTION OF THE DRAWINGS

FIGS. 1A and 1B illustrate the percentage of granulocytes expressing defensin. The results show that approximately 95% of these granulocytes express defensin.

FIG. 2A-B illustrates granulocytes expressing intracellular defensin. FIG. 2A shows the granulocyte population identified and gated based on light side scatter and signals generated from the CD15-FITC conjugate. FIG. 2B shows the percentage of the granulocyte population identified in FIG. 2A expressing intracellular defensin. Results show that approximately 79% of granulocytes expressed defensin compared to the isotype control.

FIG. 3 illustrates permeabilized and unpermeabilized granulocytes binding to defensin-specific antibody. Control cells were permeabilized and either no antibody was added or an isotype control antibody was added. Only the permeabilized granulocytes bound to defensin-specific antibody. None of the controls or unpermeabilized cells bound to defensin-specific antibody.

FIG. 4A-D illustrates CD3+/CD8+ T cells identified in a whole blood sample expressing intracellular defensin. FIGS. 4A and 4B show the relative percentage of CD3+/CD8+ T cells expressing intracellular defensin in cells in unactivated and activated cells on day 1, respectively. FIGS. 4C and 4D show the relative percentage of CD3+/CD8+ T cells expressing intracellular defensin in cells in unactivated and activated cells on day 2, respectively.

### DETAILED DESCRIPTION OF THE INVENTION

The present invention relates to a method for determining defensin expression in a multiplicity of leukocyte subpopulations.

As used herein, “leukocyte subpopulations” refers to cell types which make up the leukocyte population. In general, these comprise lymphocytes, monocytes, and granulocytes. Granulocytes further comprise eosinophils, basophils, and neutrophils and lymphocytes further comprise T cells, B cells, and natural killer (NK) cells. Monocytes may comprise immature dendritic cells, monocyte-derived macrophages and mature monocyte-derived dendritic cells. A “multiplicity of leukocyte subpopulations” refers to any two or more different leukocyte cell types. Thus, a sample comprising a “multiplicity of leukocyte subpopulations” may comprise all leukocyte cell types, such as those found in a whole blood sample. A sample may also be partially purified by methods known in the art, in which a “multiplicity of leukocyte subpopulations” comprises, for example, lymphocytes, monocytes, eosinophils, and neutrophils. A sample may also be, for example, lymphatic fluid, lymphatic cells from a tissue biopsy, or a Ficoll-purified lymphocyte population comprising T cells, B cells, and NK cells.

A sample may be obtained from blood, lymph, bone marrow, lymph node, or any other source which comprises a multiplicity of leukocyte subpopulations.

The leukocyte subpopulations may be identified and distinguished by flow cytometry. Flow cytometry is an analytical method well known in the art. For a review, see Ormerod (ed.), 1997, Flow Cytometry: A Practical Approach, Oxford Univ. Press; Jaroszeski et al. (eds.), 1997, Flow Cytometry Protocols in Methods in Molecular Biology No. 91, Humana Press; Practical Flow Cytometry, 3^{rd} ed., 1995, Wiley-Liss.

Flow cytometers useful for carrying out the present invention include conventional flow cytometers performing only flow cytometry. Such stand-alone flow cytometers are made by Beckman Coulter, Toa Medical Electronics, Cytomation, Bio-Rad, and Becton Dickinson. Other flow cytometers useful for carrying out the present invention include those integrated into other instruments, such as conventional hematology instruments. Instruments combining hematology and flow cytometry are disclosed in U.S. Pat. No. 6,228,652, 5,631,165, and 5,565,499, and are commercially available from Beckman Coulter and Toa Medical Electronics (HST Series).

Flow cytometry allows the rapid measurement of light scatter and fluorescence emission produced by suitably illuminated cells or particles. The cells or particles produce signals when they pass individually through a beam of light. Each particle or cell is measured separately and the output represents cumulative individual cytometric characteristics.

An important analytical feature of flow cytometers is their ability to measure multiple cellular parameters. The scattered light and fluorescence emissions of each particle are collected by detectors and sent to a computer, where the distribution of the population with respect to the different parameters is represented. Scattered light collected in the same direction as the incident light is related to cell size, and scattered light collected at an angle of 90° indicates particle complexity. Particle complexity is related to cell surface characteristics and the number and type of organelles present in the cell. Size and complexity are considered intrinsic parameters since they can be obtained without having to stain the sample. Using standard light scatter techniques, specific leukocyte cell types such as monocytes, lymphocytes, and granulocytes may be identified (Jackson et al., 1986, Manual of Clinical Laboratory Immunology, 3^{rd} Edition, p. 226-235). Furthermore, specific population of cells can be “gated” and analyzed separately from the remainder of the population. Using such gating techniques, flow cytometers are able to physically separate cell subsets based on their size and granularity.

To obtain additional or more specific information, samples may be stained using different fluorochromes. Fluorochromes may be classified according to their mechanism of action: fluorochromes such as fluorescein isothiocyanate (FITC), propidium iodide (PI), and Nile Red, whose fluorescence increases with binding to specific cell compounds such as proteins, nucleic acids, and lipids, respectively; those whose fluorescence depends on cellular physiological parameters such as pH and membrane potential; and those whose fluorescence depends on enzymatic activity such as esterases, peroxidases, and peptidases. Examples of fluorochromes that may be used in the instant invention are listed in Table 1. The list is not intended to be exhaustive and other fluorochromes known in the art may be used.

TABLE 1
Examples of Fluorochromes For Use in Flow Cytometry

| | Excitation | Emission | |
|---|---|---|---|
| | wavelength | wavelength | |
| Fluorochrome | (λₘₐₓ) (nm) | (λₘₐₓ) (nm) | Applications |
| Fluorescein | 495 | 525 | Protein |
| isothiocyanate (FITC) | | | |
| Texas Red | 580 | 620 | Protein |
| (sulforhodamine | | | |
| isothiocyanate) | | | |
| Oregon Green | 496 | 526 | Protein |
| isothiocyanate | | | |
| Phycoerythrin (PE) | 545 | 576 | Protein |
| Allophycocyanin | 650 | 660 | Protein |
| (APC) | | | |
| TOTO-3 | 642 | 660 | DNA, RNA |
| SYTOX Green | 504 | 525 | DNA, RNA |
| PI | 536 | 625 | DNA, RNA |
| Ethidium bromide | 510 | 595 | DNA, RNA |
| Hoechst 33258/33342 | 340 | 450 | DNA (GC pairs) |
| SYTO 13 | 488 | 509 | DNA, RNA |
| Mithramycin | 425 | 550 | DNA |
| Pyronine Y | 497 | 563 | RNA |
| Indo-1 | 340 | 398-485 | Ca²⁺ mobilization |
| Fura-2 | 340 | 549 | Ca²⁺ mobilization |
| Fluor-3 | 469 | 545 | Ca²⁺ mobilization |
| BCECF | 460-510 | 520-610 | pH |
| SNARF-1 | 510 | 587-635 | pH |
| DIOC₆(3) | 484 | 501 | Membrane potential |
| Oxonol [DiBAC₄(3)] | 488 | 525 | Membrane potential |
| Rhodamine 123 | 507 | 529 | Membrane potential |
| | | | (mitochondria) |
| Nile Red | 490-550 | 540-630 | Lipids |

The fluorochromes may be conjugated to antibodies, proteins, polypeptides, peptides, or nucleotide probes which specifically bind to antigens, proteins, polypeptides, peptides, polysaccharides, DNA, or RNA sequences. Thus, binding of an antibody, protein, polypeptide, peptide, or nucleotide probe to an antigen, protein, polypeptide, peptide, polysaccharide, DNA, or RNA may be detected by measuring the signal generated from a fluorochrome by flow cytometry. Detection of a signal may indicate binding, whereas lack of detection of a signal may indicate lack of binding. In the present invention, at least one cell-distinguishing antibody may be used to identify and distinguish leukocyte subpopulations and at least one defensin-specific antibody may be used to identify cells which express defensin.

In one embodiment of the present invention, at least one cell-distinguishing antibody may be conjugated to a fluorochrome. In another embodiment of the present invention, at least one defensin-specific antibody may be conjugated to a fluorochrome. If the cell-distinguishing antibody or defensin-specific antibody is not conjugated to a fluorochrome, a secondary antibody conjugated to a fluorochrome and specific for the cell-distinguishing antibody or for the defensin-specific antibody may be used. In a further embodiment of the present invention, at least one cell-distinguishing antibody and at least one defensin-specific antibody are conjugated to different fluorochromes. When a plurality of cell-distinguishing antibodies are used, the antibodies may be conjugated to different fluorochromes, or specifically bind to secondary antibodies conjugated to different fluorochromes.

As used herein, “cell-distinguishing antibody” refers to any antibody that may be used, alone or in combination, to facilitate the identification of leukocyte cell types. These may include, among others, antibodies specific for CD3, CD4, CD8, CD14, CD15, CD19, CD20, CD56, CD66a, CD66b, CD69, CDw125, or CD154. Other cell-distinguishing antibodies are known in the art. These cell-distinguishing antibodies typically bind to the surface of a cell, although other cell-distinguishing antibodies which bind inside the cell may be used.

A specific leukocyte cell type may be identified by either positive selection, i.e. selecting for cells that bind to one or more cell-distinguishing antibodies, or by negative selection, i.e. selecting for cells that fail to bind to one or more cell-distinguishing antibodies. For example, cytotoxic T cells may be identified by positively by selecting for cells that bind to both anti-CD3 and anti-CD8 antibodies. A combination of positive and negative selection may also be used.

A specific leukocyte cell type may also be identified according to a combination of light scatter and fluorescence generated from at least one cell-distinguishing antibody. For example, granulocytes may be identified by detecting cells which fluoresce because of binding to an anti-CD15 antibody conjugated to FITC and which also exhibit light side scatter. Other combinations of light scatter and fluorescence are also feasible in identifying cell types and are readily determinable by one skilled in the art.

As used herein, “defensin-specific antibody” refers to any antibody that may be used, alone or in combination, to detect the presence of defensin protein. The defensin-specific antibody may bind specifically to any one of alpha, beta, or theta defensins. In an embodiment of the present invention, the defensin-specific antibody binds to defensin inside a cell. In order for the defensin-specific antibody to gain access to defensin inside a cell, a cell may require permeabilization prior to the binding of the antibody to defensin inside a cell. Ideally, the permeabilizing agent creates apertures in the cell membrane without affecting the gross morphology of the cell such that flow cytometric light scattering characteristics of the cell are not affected. Such methods of permeabilizing cells are well known in the art. Moreover, the cell may be fixed prior to or during permeabilization to maintain the integrity of the cell. Methods of fixation are also well known in the art. An example of a fixation/permeabilizing agent is INTRAPREP™ (Beckman Coulter, Inc.) which comprises 5.5% v/v formaldehyde as a fixation reagent and a phosphate buffered saline (PBS)-saponin-based permeabilization reagent.

To determine the expression of intracellular defensin, the sample comprising a multiplicity of leukocyte subpopulations may be combined with at least one cell-distinguishing antibody, permeabilized, and then combined with at least one defensin-specific antibody. In another embodiment, the sample may be permeabilized, and then combined with at least one cell-distinguishing antibody and at least one defensin-specific antibody, sequentially (with at least one incubation step between the addition of the antibodies) or simultaneously (with virtually no incubation step between the addition of the antibodies). In yet another embodiment of the present invention, the sample may be combined with at least one cell-distinguishing antibody and/or defensin-specific antibody and then permeabilized.

The cell types expressing defensin may be determined by identifying at least one leukocyte subpopulation by any of the methods described above and measuring the signals generated from one or more fluorochromes that indicate the binding of a defensin-specific antibody to the leukocyte subpopulation.

The signals generated from a defensin-specific antibody may be compared to a reference value to determine if at least one leukocyte subpopulation expresses elevated or reduced level of defensin. A reference value may provide the signal from a defensin-specific antibody in at least one leukocyte subpopulation of a normal patient. The reference value may also provide the signal from a defensin-specific antibody in at least one leukocyte subpopulation which has been contacted with a cell activator. A cell activator refers to any substance which induces a change in any phenotypic or functional aspect of a cell. For example, a cell activator may up-regulate the production of defensin in a cell or may activate a T cell to engage in cytotoxic activity. A cell activator may be an antigen-specific cell activator, which is an antigen which activates only cells specific for the antigen. Examples of antigen-specific cell activators include cytomegalovirus (CMV) peptides (Frankenberg et al., 2002, Virology 295:208-216) and HIV proteins/peptides (Betts et al., 2001, Immunol. Lett. 79:117-125). Other antigen-specific activators are known in the art. Alternatively, a cell activator may be a polyclonal activator which activates virtually all cells indiscriminately. Examples of polyclonal activators include phytohaemagglutinin (PHA), bacterial lipopolysaccharide (LPS), and Staphylococcal enterotoxin A (SEA). Other polyclonal activators are known in the art.

The reference value may also serve as a negative or positive control. The negative control may be a sample combined with one or more irrelevant antibodies that would not usually result in binding to any of the cells. The negative control may also be one in which no antibody is added to the sample. If the test sample is combined with an unconjugated primary antibody and a secondary antibody conjugated to a fluorochrome, the negative control may comprise a sample in which only the unconjugated primary antibody is added, or in which only the secondary antibody is added.

The positive control may comprise a sample known to bind to certain antibodies. A positive control may thus, for example, be a sample comprising granulocytes known to bind to a defensin-specific antibody.

The disclosed method is useful for generating a profile of defensin expression in specific cell types from a given sample. A growing body of evidence suggests that changes in gene and protein expression may correlate with a disease state. Protein expression analysis of cells should allow the identification of proteins whose expression is altered in a given illness. Moreover, protein expression analysis should allow the identification of specific cell types in which expression of certain proteins have been altered in the disease state. A protein expression profile providing specific cell types and their expression of certain proteins may therefore be useful in diagnosing diseases and illnesses.

Additionally, such protein expression profiles may be useful in identifying substances effective in treating diseases and illnesses. Combining information about altered expression in a disease state with the changes that result from treatment with a substance would provide valuable information about the classes of substances that may be effective in treating a certain disease or illness. Protein expression profiling therefore may be an important tool in diagnosing diseases or illnesses as well as in identifying substances for treatment of such diseases or illnesses. In addition, the use of image capture with the flow cytometer enables the visual analysis of the morphology of the defensin expressing cells. The use of images of the defensin expressing cells might enable the definitive identification of the disease cell with a corresponding correlation to a disease state. For example, with image analysis coupled with the light scatter and fluorescent detection of the defensin producing cells on the flow cytometer, one would be able to evaluate the cell image and determine from the cell morphology whether it is a disease cell, such as from a type of cancer or an autoimmune disease, and determine a correlation of the progression of the disease.

The present invention provides a method for determining the defensin-expression profile in a multiplicity of leukocyte subpopulations. The method comprises identifying a multiplicity of leukocyte subpopulations by any of the methods described above and measuring the signal indicating binding of the defensin-specific antibody to the multiplicity of subpopulations. A defensin-expression profile provides the relative number of cells expressing defensin in the leukocyte subpopulations detected in a sample. Alternatively, a defensin-expression profile provides cell types that express defensin, and/or those that do not. A defensin-expression profile may be determined for at least two leukocyte subpopulations detected in a sample. In another embodiment of the present invention, the defensin-expression profile may be determined for three or more leukocyte subpopulations detected in a sample. In a further embodiment, the defensin-expression profile may be determined for each leukocyte subpopulation detected in a sample. Moreover, the defensin-expression profile from a test sample may be compared to a reference profile obtained from a reference sample. A reference sample may be obtained from a normal patient, a diseased patient, cells which had been combined with a cell activator, or may be cells serving as negative or positive controls, as described above.

As explained above, the defensin-expression profile may also be useful for screening substances that may be effective in treating diseases and illnesses. A test substance may be any natural or synthetic chemical, organometallic compound, or any other organic compounds. The test substance may also be a natural, synthetic, or recombinant nucleic acid, peptide, polypeptide, protein, or polysaccharide. Examples of such substances include cytokines, small molecules, drugs, and hormones. When screening for substances, the defensin-expression profile may be obtained from a sample combined with a test substance. The defensin-expression profile may be compared to a defensin-expression profile of a sample that has not been combined with the test substance to determine whether defensin expression can be up-regulated or down-regulated in certain cell types.

Defensin-expression profiles may be combined with other immunological profiles to aid in diagnosing and treating diseases or illnesses because other immunological responses may influence the expression of defensin in specific cell types. Examples of other immunological profiles include an intracellular cytokine-expression profile, a cytokine-binding profile, a peptide-MHC binding profile, an antigen-specific binding profile, a cell proliferation profile, or an immune activation profile. An intracellular cytokine-expression profile provides cell types which express a certain cytokine and is useful for determining whether production of that cytokine is up-regulated or down-reguated in certain cell populations. A cytokine-binding profile provides cell types which express cytokine receptors and which are capable of being regulated by certain cytokines. A peptide-MHC binding profile provides T cell types which are capable of recognizing the specific peptide bound to the major histocompatibility complex (MHC). A peptide-MHC binding profile is useful for determining whether certain T cells in a sample are capable of recognizing and clearing an antigen from a system. An antigen-specific binding profile provides B cells which are capable of recognizing and binding a specific antigen via the B cell receptor. A cell proliferation profile provides cell types which are proliferating as an indication of immune activation or differentiation. An immune activation profile provides cell types that have been activated to effect an immune response.

To obtain such other immunological profiles, a fluorochrome may be conjugated to a cytokine-specific antibody, a cytokine, a peptide-MHC complex, an antigen, a reagent for detecting cell proliferation or an antibody indicative of immune activation. If any of these proteins or antibodies are combined together in a sample for simultaneous detection, the proteins or antibodies are conjugated to different fluorochromes. If these immunological parameters are assessed separately from each other, the proteins or antibodies may be conjugated to the same or different fluorochromes.

Examples of cytokine-specific antibodies and cytokines include antibodies specific for the cytokines IL-2, IL-3, IL-4, IL-5, IL-10, tumor necrosis factor (TNF) alpha and beta, granulocyte-macrophage colony-stimulating factor (GM-CSF), granulocyte-colony stimulating factor (G-CSF) and transforming growth factor (TGF). Other antibodies to cytokines and cytokines are known in the art.

Peptide-MHC complexes may be produced by methods known in the art. For example, specific peptide-MHC complexes may be produced in yeast (see e.g., Brophy et al., 2003, J. Immunol. Methods 272:235-246); by viral vectors (see e.g., Kawana-Tachikawa et al., 2002, J. Virol. 76:11982-11988); by expression of a specific peptide-MHC complex in mammalian cell (see e.g., Mottez et al., 1995, J. Exp. Med. 181:493-502); or by producing MHC in E. coli and complexing it with single antigenic peptides (see e.g., Graboczi et al., 1992, Proc. Natl. Acad. Sci. USA 89:3429-3433). Other methods of producing peptide-MHC complexes are known in the art.

Antigens recognized by B cells may be any antigen, and may include “self” or “non-self” antigens. Flow cytometry has been used to detect memory or plasma B cells which bind antigens and is described in, for example, Schittek et al., 1990, Nature 346:749-751, and Leyendeckers et al., 1999, Eur. J. Immunol. 29: 1406-1417.

Reagents for detecting cell proliferation include any reagent useful for detecting cell proliferation. These include fluorescent labels which label cellular proteins, such as carboxyfluorescein-diactetate-succinimidylester (CFDA). Dilution of the label is indicative of proliferation. Other reagents include, for example, bromodeoxyuridine (BrdU), a pyrimidine analogue which is incorporated into DNA of proliferating S-phase cells or proliferating cells expressing cell cycle-associated proteins such as cyclins. A monoclonal antibody against BrdU may be used to detect incorporated BrdU (Pechhold et al., 1998, Methods in Microbiology 25:59-78). Other reagents useful for detecting cell proliferation are known in the art.

Examples of antibodies indicative of immune activation include antibodies specific for CD25, CD45RA, CD45RO, CD54, CD38, HLA-DR, Leu8, and CD23. These may include antibodies to cytokine receptors, such as antibodies to receptors of cytokines interferons (IFN α, β, and γ), IL-1, IL-2, II-3, IL-4, IL-5, IL-6, IL-7, IL-9, IL-10, IL-12, IL-13, IL-15, IL-16, IL-17, tumor necrosis factor (TNF) alpha and beta, granulocyte-macrophage colony-stimulating factor (GM-CSF), granulocyte-colony stimulating factor (G-CSF) and transforming growth factor (TGF). Other cytokine receptors are known in the art. These surface molecules may be either up-regulated or down-regulated upon immune activation. For example, CD45RA and Leu8 are down-regulated on T cells following activation, whereas HLA-DR (MHC class II) and CD25 (IL-2 receptor) are increased upon T cell activation. Other antibodies indicative of immune activation are well known in the art.

The intracellular cytokine-expression profile may be determined by combining a sample comprising a multiplicity of leukocyte subpopulations with at least one defensin-specific antibody and at least one cytokine-specific antibody which specifically binds to a cytokine inside a cell. The cells in the sample may be permeabilized as described above. The signals from the cytokine-specific antibody and defensin-specific antibody are measured in a multiplicity of leukocyte subpopulations identified by any of the methods described above. An intracellular cytokine-expression profile providing the relative number of cells expressing intracellular cytokine and defensin in the leukocyte subpopulations is generated. The intracellular cytokine-expression profile may be compared to a reference profile. A reference profile is obtained from a reference sample. A reference sample may be obtained from a normal patient, a diseased patient, cells which had been combined with a cell activator, or may be cells serving as negative or positive controls, as described above.

Similarly, the cytokine-binding profile may be determined by combining a sample comprising a multiplicity of leukocyte subpopulations with at least one defensin-specific antibody and at least one cytokine which specifically binds to the surface of a cell. The cells in the sample may be permeabilized as described above. The signals from the cytokine and defensin-specific antibody are measured in a multiplicity of leukocyte subpopulations identified by any of the methods described above. A cytokine-binding profile providing the relative number of cells binding cytokine and expressing defensin in the leukocyte subpopulations is generated. The cytokine-binding profile may be compared to a reference profile. A reference profile is obtained from a reference sample. A reference sample may be obtained from a normal patient, a diseased patient, cells which had been combined with a cell activator, or may be cells serving as negative or positive controls, as described above.

The peptide-MHC binding profile may be determined by combining a sample comprising a multiplicity of leukocyte subpopulations with at least one defensin-specific antibody and at least one peptide-MHC complex which specifically binds to the T cell surface. The cells in the sample may be permeabilized as described above. T cells may be identified by any of the methods described above. In one embodiment, T cells may be identified by identifying cells which bind to both antibodies against CD3 and CD8. In another embodiment, T cells may be identified by identifying cells which bind to both antibodies against CD3 and CD4 or to antibodies against CD4 and CD8. The signals from the peptide-MHC complex and defensin-specific antibody are measured in the T cells. A peptide-MHC binding profile providing the relative number of cells binding peptide-MHC and expressing defensin in the T cell subpopulations is generated. The peptide-MHC binding profile may be compared to a reference profile. A reference profile is obtained from a reference sample. A reference sample may be obtained from a normal patient, a diseased patient, cells which had been combined with a cell activator, or may be cells serving as negative or positive controls, as described above.

The immune activation profile may be determined by combining a sample comprising a multiplicity of leukocyte subpopulations with at least one defensin-specific antibody and at least one antibody indicative of immune activation which specifically binds to the surface of a cell. The cells in the sample may be permeabilized as described above. The signals from the antibody indicative of immune activation and defensin-specific antibody are measured in a multiplicity of leukocyte subpopulations identified by any of the methods described. An immune activation profile providing the relative number of cells binding to the antibody indicative of immune activation and expressing defensin in the leukocyte subpopulations is generated. The immune activation profile may be compared to a reference profile. A reference profile is obtained from a reference sample. A reference sample may be obtained from a normal patient, a diseased patient, cells which had been combined with a cell activator, or may be cells serving as negative or positive controls, as described above.

An antigen specific binding-profile may be determined by combining a sample comprising a multiplicity of leukocyte subpopulations with at least one defensin-specific antibody and at least one antigen. The cells in the sample may be permeabilized as described above. B cells may be identified by any of the methods described above. For example, B cells may be identified by identifying cells which bind to both antibodies against CD19 and CD20. In one embodiment, memory B cells may be identified by identifying cells which bind to antibodies against CD19, MHC class II, and to an activation marker such as CD27. In another embodiment, plasma cells may be identified by identifying cells which binds to an antibody against and CD19 but not to an antibody against MHC class II. Plasma cells from bone marrow may be identified by detecting cells which bind antibodies to CD38 and CD138 (Rawstron et al., 2002, Blood 100:3095-3100). The signals from the antigen and defensin-specific antibody are measured in the B cells and an antigen-specific binding profile providing the relative number of cells binding antigen and expressing defensin in the B cells is generated. The antigen-specific binding profile may be compared to a reference profile. A reference profile is obtained from a reference sample. A reference sample may be obtained from a normal patient, a diseased patient, cells which had been combined with a cell activator, or may be cells serving as negative or positive controls, as described above.

A proliferation profile may be determined by combining a sample comprising a multiplicity of leukocyte subpopulations with at least one defensin-specific antibody and at least one reagent for detecting cell proliferation. The cells may be permeabilized as described above. The signals from the reagent for detecting cell proliferation and defensin-specific antibody are measured in a multiplicity of leukocyte subpopulations identified by any of the methods described above. A proliferation profile providing the relative number of cells proliferating and expressing defensins in the leukocyte subpopulations is generated. The proliferation profile may be compared to a reference profile. A reference profile is obtained from a reference sample. A reference sample may be obtained from a normal patient, a diseased patient, cells which had been combined with a cell activator, or may be cells serving as negative or positive controls, as described above.

The present invention also provides a kit for determining cell types expressing defensin and for determining a defensin-expression profile. The kit comprises at least one cell-distinguishing antibody and at least one defensin-specific antibody. As described above, at least one cell-distinguishing antibody may be conjugated to a fluorochrome. In another embodiment of the present invention, at least one defensin-specific antibody is conjugated to a fluorochrome. In a further embodiment, at least one cell-distinguishing antibody is conjugated to a fluorochrome and at least one defensin-specific antibody is conjugated to a different fluorochrome. If the cell-distinguishing antibody or defensin-specific antibody is not conjugated to a fluorochrome, the kit may also comprise a secondary antibody conjugated to a fluorochrome and specific for the cell-distinguishing antibody or for the defensin-specific antibody. In another embodiment, a plurality of cell-distinguishing antibodies are included in the kit. The plurality of cell-distinguishing antibodies may be conjugated to different fluorochromes, or bind specifically to secondary antibodies conjugated to different fluorochromes.

The kit may further comprise a permeabilizing agent and/or a fixing reagent such as those described above. The kit may also comprise any one or more of the following: a cytokine-specific antibody, a cytokine, an antibody to a cytokine receptor, a peptide-MHC complex, a reagent for detecting cell proliferation, and an antibody indicative of immune activation.

The present invention is illustrated by the following Examples, which are not intended to be limiting in any way.

### EXAMPLES

### Example 1

### Intracellular Defensin Expression in Granulocytes Identified by Light Scatter

Whole blood obtained from a normal patient was fixed and permeabilized with INTRAPREP (Beckman Coulter, Inc.). A commercially-available alpha defensin-specific monoclonal antibody, DEF3 (Accurate Chemical) or D21 (Cell Sciences), was then added to the sample and allowed to bind to defensin inside the cells. Because DEF3 nor D21 are conjugated to a fluorochrome, an anti-IgG secondary antibody conjugated to a fluorochrome was then added to the cells. Following binding of the secondary antibody to the defensin-specific antibody, the cells were washed and analyzed by flow cytometry.

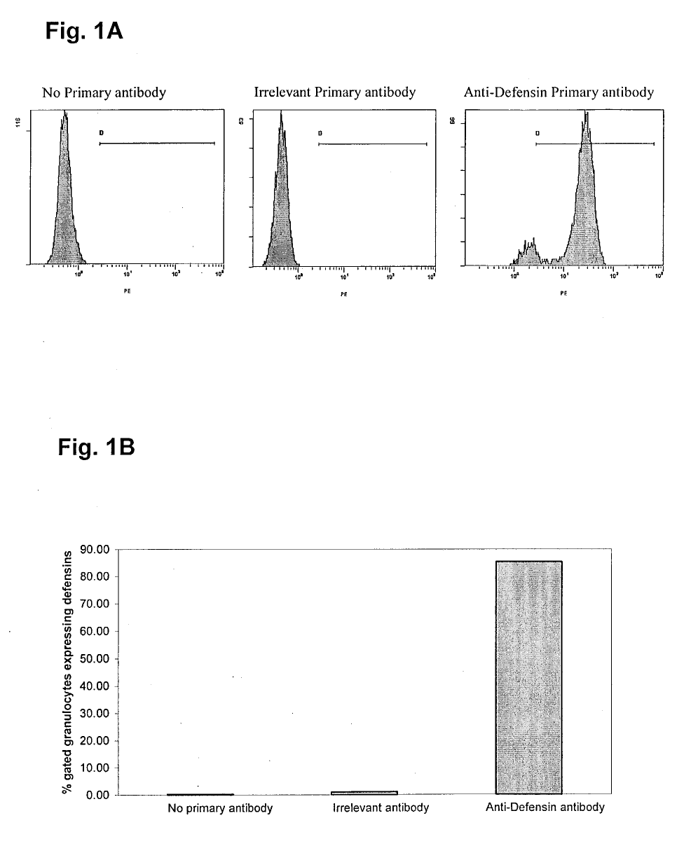

The granulocyte population was identified based on forward versus side scatter. The percentage of cells binding to the defensin-specific antibody was determined in this granulocyte population by comparing the signals obtained from a granulocyte population in which no defensin-specific antibody was added and from a granulocyte population in which an irrelevant primary antibody was added. The percentage of granulocytes expressing defensin was determined as shown in FIG. 1A and plotted in FIG. 1B. The results show that approximately 95% of these granulocytes express defensin.

### Example 2

### Intracellular Defensin Expression in Granulocytes Identified by a Cell-Distinguishing Antibody

Whole blood obtained from a normal patient was first combined with an anti-CD15 conjugated to FITC. The cells were then fixed and permeabilized with INTRAPREP (Beckman Coulter, Inc.). An alpha defensin-specific antibody, DEF3 or D21, was added to the sample and allowed to bind to defensin inside the cells. A secondary antibody specific for the defensin antibody and conjugated to phycoerythrin (PE) was added to the sample. The cells were washed and analyzed by flow cytometry.

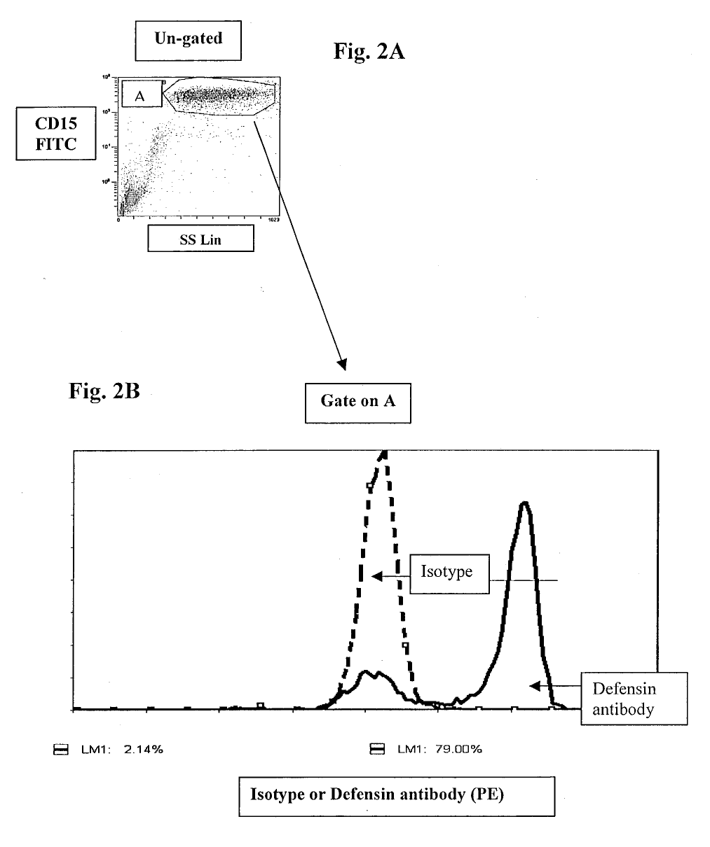

The granulocyte population was identified and gated based on light side scatter and signals generated from the FITC conjugate as shown in FIG. 2A. The gated cell population was analyzed for signals generated from the PE-conjugated antibody. The percentage of cells binding to defensin-specific antibody was determined in this granulocyte population by comparing the signals obtained from a granulocyte population in which an isotype of the defensin-specific antibody was added (FIG. 2B). The isotype control was not expected to bind to the cells and therefore served as a negative control. FIG. 2B shows that approximately 79% of CD15-expressing granulocytes expressed defensin.

### Example 3

### Intracellular Defensin Expression in Permeabilized Granulocytes

In this Example, intracellular defensin expression was detected only in granulocytes identified according to the method described in Example 2 when the cells were permeabilized. This example therefore confirms that the binding of defensin-specific antibody reflects intracellular defensin expression.

Whole blood obtained from a normal patient was first combined with an anti-CD15 conjugated to FITC as in Example 2. The cells were either fixed and permeabilized with INTRAPREP (Beckman Coulter, Inc.) or left unpermeabilized. Alpha defensin-specific antibody, DEF3 or CD21, was added to the samples. A secondary antibody specific for the defensin antibody and conjugated to phycoerythrin (PE) was then added to the sample. Control cells were permeabilized as above and either no antibody was added or an isotype control antibody was added. The cells were washed and analyzed by flow cytometry.

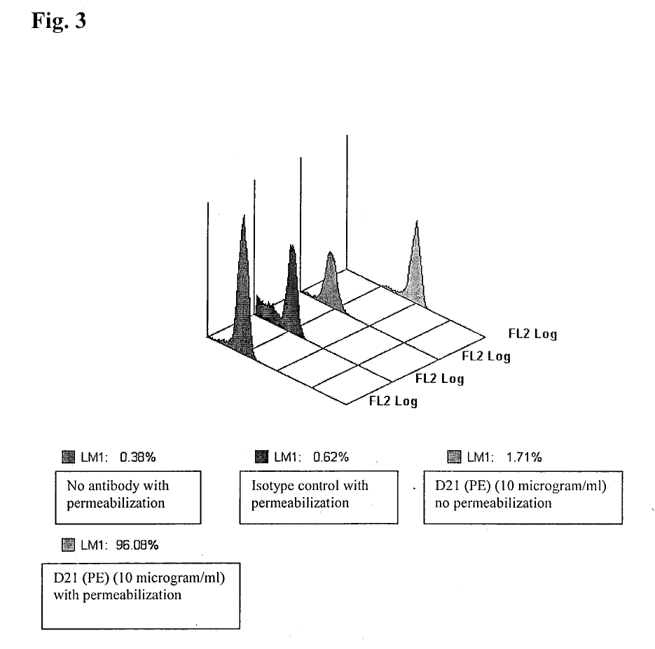

As shown in FIG. 3, only the permeabilized granulocytes bound to defensin-specific antibody. None of the controls or unpermeabilized cells bound to defensin-specific antibody.

### Example 4

### Intracellular Defensin Expression in T Cells

In this Example, cells in a whole blood obtained from a normal patient was activated for one or two days by adding Staphylococcus aureas enterotoxin B (SEB) and an anti-CD28 antibody. Unactivated samples were prepared alongside the activated samples. Anti-CD3 and anti-CD8 antibodies conjugated to different fluorochromes were then added to the samples and the cells were fixed and permeabilized with INTRAPREP (Beckman Coulter, Inc.). An alpha defensin-specific antibody, DEF3 or D21, was added to the samples and allowed to bind to defensin inside the cells. A secondary antibody specific for the defensin antibody and conjugated to phycoerythrin (PE) was added to the samples. The cells were washed and analyzed by flow cytometry.

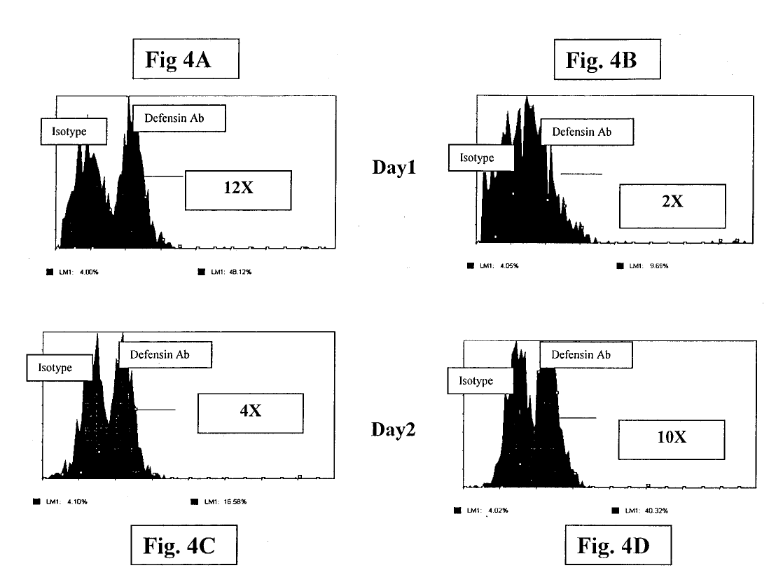

CD3+/CD8+ T cells were identified and the percentage of those T cells expressing intracellular defensin was determined. FIGS. 4A and 4B show the relative percentage of CD3+/CD8+ T cells expressing intracellular defensin in unactivated and activated cells on day 1, respectively. 48% of unactivated T cells express defensin, whereas about 10% of activated T cells express defensin on day 1. On day 2, about 17% of unactivated T cells express defensin (FIG. 4C) and 40% of activated T cells express defensin (FIG. 4D).

### Example 5

### Intracellular Defensin-Expression Profile

Utilizing the methods described in the above Examples, an intracellular defensin-expression profile of various leukocyte cell types may be generated.

A whole blood sample is combined with various cell-distinguishing antibodies, such as CD15 (for monocytes and granulocytes), CD3 (for T cells), CD8 (for T cells), and CD4 (for T cells), all conjugated to different fluorochromes. The sample is then permeabilized with INTRPREP (Beckman Coulter, Inc.) and a defensin-specific antibody such as D21 is added. A secondary antibody specific for D21 and conjugated to a fluorochrome different from those conjugated to the cell-distinguishing antibody is added to the cells. The cells are washed and analyzed by flow cytometry.

Granulocytes are identified according to the method described in Example 2, CD3+/CD8+ T cells are identified by selecting for cells binding to both CD3 and CD8 antibodies, and CD3+/CD4+ T cells are identified by selecting for cells binding to both CD3 and CD4 antibodies. All three cell populations are analyzed for binding to the defensin-specific antibody. The percentage of cells expressing defensin in each cell population is determined, creating an intracellular defensin-expression profile comprising of granulocytes, CD3+/CD8+ T cells, and CD3+/CD4+ T cells. Additional cell populations may be analyzed for intracellular defensin expression to provide a comprehensive intracellular defensin-expression profile.

### Example 6

### Intracellular Defensin-Expression Profile and Other Immunological Profiles

It would be useful to identify other immunological responses associated with defensin expression in specific cell types because other immunological responses may influence the expression of defensin. Intracellular cytokine expression may be one of these factors.

In a sample prepared as in Example 5, an antibody against a cytokine such as IL-2 and conjugated to yet another fluorochrome may be added the permeabilized sample. Each cell population identified and analyzed for intracellular defensin expression is also analyzed for binding to the anti-cytokine antibody. The percentage of cells expressing intracellular cytokine in each cell population is determined by flow cytometry, creating an intracellular defensin-expression profile and an intracellular cytokine-expression profile. Other immunological profiles may be generated in this manner.

### Example 7

### Screening for Therapeutic Substances Affecting Intracellular Defensin Expression

Substances up-regulating or down-regulating intracellular defensin may be screened using the intracellular defensin expression profile obtained as in Example 5.

A sample obtained from a normal or diseased patient is used to generate an intracellular defensin-expression profile in the presence and absence of a defensin-modulating substance such as IL-1 beta and TNF-alpha (Tsutsumi-Ishii et al., 2003, J. Immunol. 170:4226-4336) or heat/chemically inactivated bacteria. The two defensin-expression profiles of various cell types are compared to determine which cell types are affected in defensin expression by the substance. If the substance is shown to increase defensin expression in certain cell types known to combat certain infections, that substance may be useful in treating that infection. On the other hand, if a substance is shown to down-regulate defensin expression in cells known to increase an allergic response, that substance may be useful in treating allergies.

The specification is most thoroughly understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with a true scope and spirit of the invention being indicated by the following claims.

What is claimed is:

1. A method for determining cell types expressing intracellular defensin comprising,
(a) providing a sample comprising a multiplicity of leukocyte subpopulations;
(b) combining the sample with at least one defensin-specific antibody, wherein said defensin-specific antibody binds to defensin inside a cell;
(c) combining the sample with at least one cell-distinguishing antibody, wherein said cell-distinguishing antibody binds to the cell surface of at least one leukocyte subpopulation;
(d) identifying at least one leukocyte subpopulation by measuring the signal from said cell-distinguishing antibody bound to said at least one leukocyte subpopulation; and
(e) measuring the signal from said defensin-specific antibody in said at least one leukocyte subpopulation identified in step (d).

2. The method of claim 1, wherein at least one cell-distinguishing antibody is CD3, CD4, CD8, CD14, CD15, CD19, CD20, CD56, CD66a, CD66b, CD69, CDw125, or CD154.

3. The method of claim 1, wherein the sample is combined with a plurality of said cell-distinguishing antibodies.

4. The method of claim 3, wherein step (e) comprises measuring by flow cytometry the signal from said defensin-specific antibody in a gated subset of cells, which are bound to at least two cell-distinguishing antibodies.

5. The method of claim 4, wherein said at least two T-cell distinguishing antibodies are CD4 and CD8, CD3 and CD4, or CD3 and CD8.

6. The method of claim 1, wherein said step (e) comprises measuring by flow cytometry the signal from said defensin-specific antibody in a gated subset of cells which (i) are bound to said at least one cell-distinguishing antibody and (ii) exhibit light scatter.

7. The method of claim 1, wherein cells are permeabilized prior to the binding of said defensin-specific antibody.

8. The method of claim 1, wherein said sample is a whole blood sample.

9. The method of claim 1, wherein said sample comprises monocytes, lymphocytes, neutrophils, and eosinophils.

10. A method for determining a defensin-expression profile of a multiplicity of leukocyte subpopulations comprising:
(a) providing a sample comprising a multiplicity of leukocyte subpopulations;
(b) combining the sample with at least one defensin-specific antibody, wherein said defensin-specific antibody binds to defensin inside a cell;
(c) identifying a multiplicity of leukocyte subpopulations;
(d) measuring the signal from said defensin-specific antibody in said multiplicity of leukocyte subpopulations identified in step (c); and
(e) generating a defensin-expression profile of said multiplicity of leukocyte subpopulations.

11. The method of claim 10, wherein said sample is a whole blood sample.

12. The method of claim 10, wherein said sample comprises monocytes, lymphocytes, neutrophils, and eosinophils.

13. The method of claim 10, wherein step (c) comprises combining the sample with at least one cell-distinguishing antibody, wherein said cell-distinguishing antibody binds to the cell surface of at least one leukocyte subpopulation.

14. The method of claim 10, further comprising determining an intracellular cytokine-expression profile of a multiplicity of leukocyte subpopulations, comprising:
further combining the sample with at least one cytokine-specific antibody,
wherein said cytokine-specific antibody binds to a cytokine inside a cell;
measuring the signal from said cytokine-specific antibody and said defensin-specific antibody by flow cytometry in said multiplicity of leukocyte subpopulations identified by step (c); and
generating an intracellular cytokine-expression profile of said multiplicity of leukocyte subpopulations.

15. The method of claim 14, wherein step (c) comprises combining the sample with at least one cell-distinguishing antibody, wherein said cell-distinguishing antibody binds to the cell surface of at least one leukocyte subpopulation.

16. The method of claim 10, further comprising determining a cytokine-binding profile of a multiplicity of leukocyte subpopulations, comprising:
further combining the sample with at least one cytokine, wherein said cytokine binds to the surface of a cell;
measuring the signal from said cytokine and said defensin-specific antibody by flow cytometry in said multiplicity of leukocyte subpopulations identified by step (c); and
generating a cytokine binding profile of said multiplicity of leukocyte subpopulations.

17. The method of claim 16, wherein step (c) comprises combining the sample with at least one cell-distinguishing antibody, wherein said cell-distinguishing antibody binds to the cell surface of at least one leukocyte subpopulation.

18. The method of claim 10, further comprising determining a peptide-MHC binding profile of T cells in a multiplicity of leukocyte subpopulations, comprising:
further combining the sample with at least one peptide-MHC complex,
wherein said peptide-MHC complex binds to the surface of a T cell;
measuring the signal from said peptide-MHC complex and said defensin-specific antibody by flow cytometry in T cells identified by step (c); and
generating a peptide-MHC binding profile of said T cells.

19. The method of claim 18, wherein step (c) comprises combining the sample with at least one T cell-distinguishing antibody, wherein said T cell-distinguishing antibody binds to the cell surface of at least one T cell subpopulation.

20. The method of claim 10, further comprising determining an immune activation profile of a multiplicity of leukocyte subpopulations, comprising:
further combining the sample with at least one antibody indicative of immune activation, wherein said antibody indicative of immune activation binds to the surface of a cell;
measuring the signal from said antibody indicative of immune activation and said defensin-specific antibody by flow cytometry in said multiplicity of leukocyte subpopulations identified by step (c); and
generating an immune activation profile of said multiplicity of leukocyte subpopulations.

21. The method of claim 20, wherein step (c) comprises combining the sample with at least one cell-distinguishing antibody, wherein said cell-distinguishing antibody binds to the cell surface of at least one leukocyte subpopulation.

22. The method of claim 10, further comprising determining an antigen-specific binding profile of B cells in a multiplicity of leukocyte subpopulations, comprising:
further combining the sample with at least one antigen, wherein said antigen binds to the surface of a B cell;
measuring the signals from said antigen and said defensin-specific antibody by flow cytometry in B cells identified by step (c); and
generating an antigen-specific binding profile of said B cells.

23. The method of claim 22, wherein step (c) comprises combining the sample with at least one B cell-distinguishing antibody, wherein said B cell-distinguishing antibody binds to the cell surface of at least one B cell subpopulation.

24. The method of claim 10, further comprising determining a cell proliferation profile of a multiplicity of leukocyte subpopulations, comprising:
further combining the sample with at least one reagent for detecting cell proliferation;
measuring the signal from said reagent and said defensin-specific antibody by flow cytometry in said multiplicity of leukocyte subpopulations identified by step (c); and
generating a cell proliferation profile of said multiplicity of leukocyte subpopulations.

25. The method of claim 24, wherein step (c) comprises combining the sample with at least one cell-distinguishing antibody, wherein said cell-distinguishing antibody binds to the cell surface of at least one leukocyte subpopulation.

26. The method of claim 13, wherein said at least one cell-distinguishing antibody is CD3, CD4, CD8, CD14, CD15, CD19, CD20, CD56, CD66a, CD66b, CD69, CDw125, or CD154.

27. The method of claim 13, wherein the sample is contacted with a plurality of said cell-distinguishing antibodies.

28. The method of claim 1 which further comprises providing an image of a cell that expresses defensin for the purpose of analyzing the morphology of said cell.

29. A kit for determining cell types expressing intracellular defensin comprising:
(a) at least one cell-distinguishing antibody; and
(b) at least one defensin-specific antibody.

30. The kit of claim 29, wherein said kit comprises a plurality of cell-distinguishing antibodies.

31. The kit of claim 29, wherein said kit further comprises a permeabilizing agent.

32. The kit of claim 29, wherein said at least one cell-distinguishing antibody is conjugated to a fluorochrome.

33. The kit of claim 29, wherein said at least one defensin-specific antibody is conjugated to a fluorochrome.

34. The kit of claim 29, wherein said at least one cell-distinguishing antibody is CD3, CD4, CD8, CD14, CD15, CD19, CD20, CD56, CD66a, CD66b, CD69, CDw125, or CD154.

35. The kit of claim 29, further comprising at least one reagent selected from:
(i) a cytokine-specific antibody;
(ii) a cytokine;
(iii) a peptide-MHC complex; and
(iv) an antigen
(v) a reagent for detecting cell proliferation; and
(vi) an antibody indicative of immune activation.

36. A kit for determining a defensin-expression profile comprising:
(a) at least one cell-distinguishing antibody;
(b) at least one defensin-specific antibody;
(c) a permeabilizing agent; and
(d) at least one reagent selected from:
(i) a cytokine-specific antibody;
(ii) a cytokine;
(iii) a peptide-MHC complex; and
(iv) an antigen
(v) a reagent for detecting cell proliferation; and
(vi) an antibody indicative of immune activation.

37. The kit of claim 36, wherein said at least one cell-distinguishing antibody is conjugated to a fluorochrome.

38. The kit of claim 36, wherein said at least one defensin-specific antibody is conjugated to a fluorochrome.

39. The kit of claim 36, wherein said at least one cell-distinguishing antibody and said at least one defensin specific antibody are conjugated to different fluorochromes.

40. The kit of claim 36, wherein said at least one cell-distinguishing antibody and said at least one reagent are conjugated to different fluorochromes.
